(19) United States
(12) Patent Application Publication
(10) Pub. No.: US 2023/0240536 A1
(43) Pub. Date: Aug. 3, 2023

(54) ENDOSCOPE CAMERA ASSEMBLY

(71) Applicant: Visionsense Ltd., Petah Tikva (IL)

(72) Inventors: Doron Aloni, Ganey Tikva (IL); Nadav Horesh, Ramat Raziel (IL); Udi Ronen, Kfar Saba (IL)

(21) Appl. No.: 17/587,187

(22) Filed: Jan. 28, 2022

(51) Int. Cl. A61B 5/00; H04N 5/225; H04N 5/232; H04N 5/265; A61B 1/04; A61B 1/00

(52) U.S. Cl. CPC A61B 5/0035; H04N 5/2252; H04N 5/2258; H04N 5/2256; H04N 5/23229; H04N 5/265; A61B 1/042; A61B 5/0086; A61B 1/00186; A61B 5/0071; H04N 2005/2255; A61B 1/00105

(57) ABSTRACT

An endoscope camera assembly includes a housing having an opening configured to couple to an endoscope and to receive a combined light entering the housing along a combined light path. The combined light includes visible light and infrared fluorescence light. The camera assembly includes a cold mirror configured to split the combined light into the visible light along a visible light path and the infrared fluorescence light along an infrared light path. The camera assembly also includes a visible light sensor configured to receive the visible light and to generate visible light image data. The camera assembly further includes an infrared sensor configured to receive the infrared fluorescence light and to generate infrared fluorescence image data.

### BACKGROUND

Medical endoscopy is increasingly employing specialized optical imaging techniques, such as fluorescence (i.e., autofluorescence and photodynamic) endoscopy, narrow band imaging and other techniques, for improved visualization and for the detection and diagnosis of diseases. Endoscopic imaging systems that provide specialized imaging modes also operate in a conventional color, or white light, endoscopy mode.

In conventional white light endoscopy, light in the visible spectral range is used to illuminate the tissue surface under observation. Light reflected by the tissue passes through a suitable lens system and is incident on an image sensor built into or attached to the endoscope. The electrical signals from the image sensor are processed into a full color video image which can be displayed on a video monitor or stored in a memory.

In fluorescence endoscopy, fluorescence excitation light excites fluorophores in the tissue, which emit fluorescence light at an emission wavelength, which is typically greater than the excitation wavelength. Fluorescence light from the tissue passes through a suitable lens system and is incident on the image sensor. The electrical signals from the image sensor are processed into a fluorescence video image which can be displayed on a video monitor, either separately or combined with the color video image.

The fluorescence excitation and emission wavelengths depend upon the type of fluorophores being excited. In the case of exogenously applied fluorophores, the band of excitation wavelengths may be located anywhere in the range from the ultraviolet (UV) to the near infra-red (NIR) and the emission wavelength band anywhere from the visible to the NIR. For fluorophores endogenous to tissue, the band of excitation and emission wavelengths are more limited (excitation from the UV to the green part of the visible spectrum, emission from the blue/green light to the NIR).

Fluorescence endoscopy may be used to identify blood vessels, cancer cells, and other tissue types. However, conventional endoscopes are limited in their interchangeability, in that optical assemblies, e.g., rod lenses, are coupled with camera units. Thus, there is a need for a camera assembly configured to be used with a variety of different endoscopes.

### SUMMARY

The present disclosure provides a camera assembly for use with an endoscope. The camera assembly operates in two channels, the visible spectrum, and IR spectrum, i.e., fluorescence mode. In particular, the camera assembly includes a white, i.e., visible, light sensor and IR light sensor that are operable in parallel, which allows for a higher frame rate. The camera assembly is configured to filter light of undesirable wavelengths while splitting IR light from white light. In addition, the camera assembly has a higher sensitivity to IR light, that is caused by the fluorescence of the tissue.

According to one embodiment of the present disclosure, a camera assembly is disclosed. The camera assembly includes a housing having an opening configured to couple to an endoscope and to receive a combined light entering the housing along a combined light path. The combined light includes visible light and infrared fluorescence light. The camera assembly includes a cold mirror configured to split the combined light into the visible light along a visible light path and the infrared fluorescence light along an infrared light path. The camera assembly also includes a visible light sensor configured to receive the visible light and to generate visible light image data. The camera assembly further includes an infrared sensor configured to receive the infrared fluorescence light and to generate infrared fluorescence image data.

According to another embodiment of the present disclosure, an endoscope assembly is disclosed. The endoscope assembly may include an endoscope having a proximal end portion, a distal end portion, and a light port configured to couple to a light source. The endoscope is configured to receive a combined light having visible light and infrared fluorescence light. The endoscope assembly also includes a camera assembly having a housing with an opening configured to couple to the distal end portion of the endoscope and to receive the combined light entering the housing along a combined light path. The camera assembly also includes a cold mirror configured to split the combined light into the visible light along a visible light path and the infrared fluorescence light along an infrared light path. The camera assembly further includes a visible light sensor configured to receive the visible light and to generate visible light image data. The camera assembly additionally includes an infrared sensor configured to receive the infrared fluorescence light and to generate infrared fluorescence image data.

According to a further embodiment of the present disclosure, an imaging system is disclosed. The imaging system includes a combined light source configured to output visible light and excitation laser light. The imaging system also includes an endoscope having a proximal end portion, a distal end portion, and a light port configured to couple to the light source. The endoscope is configured to receive a combined light having visible light and infrared fluorescence light. The imaging system further includes a camera assembly having a housing with an opening configured to couple to the distal end portion of the endoscope and to receive the combined light entering the housing along a combined light path. The camera assembly includes a cold mirror configured to split the combined light into the visible light along a visible light path and the infrared fluorescence light along an infrared light path. The camera assembly also includes a visible light sensor configured to receive the visible light and to generate visible light image data. The camera assembly further includes an infrared sensor configured to receive the infrared fluorescence light and to generate infrared fluorescence image data. The imaging system additionally includes a camera control unit coupled to the camera assembly. The camera control unit is configured to output a combined image having the visible light image data and the infrared fluorescence image data.

Implementations of the above embodiments may include one or more of the following features. According to one aspect of the above embodiment, the camera assembly may further include a notch filter disposed adjacent the opening, such that the combined light entering the opening passes through the notch filter that is configured to remove excitation laser light from the combined light. The camera assembly may also include a focus group having at least one lens. The focus group is disposed between the opening and the cold mirror and is movable along the combined light path. The notch filter may be coupled to the focus group and may be movable along with the focus group. The camera assembly may include a hot mirror disposed along the visible light path between the cold mirror and the visible light sensor. The hot mirror may be configured to transmit the visible light and to reflect the infrared fluorescence light. The hot mirror may be disposed at an incidence angle that is substantially perpendicular to the visible light path. The camera assembly may include a bandpass filter disposed along the infrared light path and between the cold mirror and the infrared sensor. The bandpass filter may be configured to transmit only the infrared fluorescence light to the infrared sensor.

### BRIEF DESCRIPTION OF THE DRAWINGS

The present disclosure may be understood by reference to the accompanying drawings, when considered in conjunction with the subsequent, detailed description, in which:

FIG. 1 is a schematic diagram of a light endoscopic imaging system according to an embodiment the present disclosure;

FIG. 2 is a perspective view of an endoscope coupled to a camera assembly according to an embodiment the present disclosure;

FIG. 3 is a side, cross-sectional view of the camera assembly of FIG. 2, according to an embodiment the present disclosure; and

FIG. 4 is a schematic view of the camera assembly of FIG. 2, according to an embodiment the present disclosure.

### DETAILED DESCRIPTION

Embodiments of the presently disclosed system are described in detail with reference to the drawings, in which like reference numerals designate identical or corresponding elements in each of the several views. In the following description, well-known functions or constructions are not described in detail to avoid obscuring the present disclosure in unnecessary detail. Those skilled in the art will understand that the present disclosure may be adapted for use with any imaging system. As used herein the term “distal” refers to that portion of the instrument, or component thereof, farther from the user, while the term “proximal” refers to that portion of the instrument, or component thereof, closer to the user.

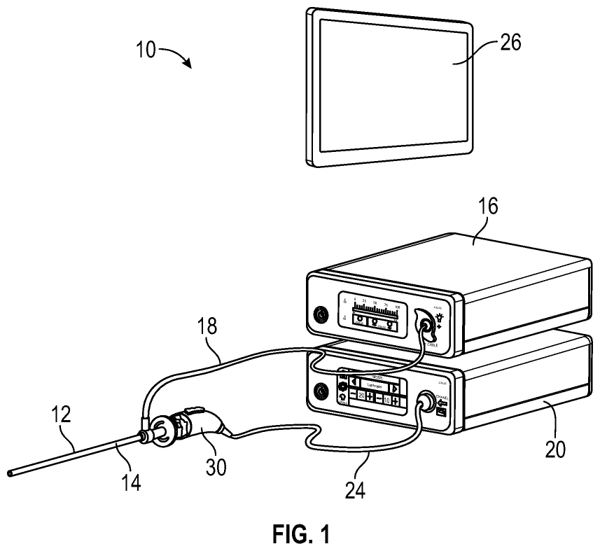

With reference to FIG. 1, an imaging system 10 is configured for combined NIR fluorescence and white light endoscopic imaging. With intraoperative usage of fluorophores from a fluorescent dye, such as indocyanine green (ICG), the imaging system 10 enables real-time visual assessment of blood vessels, lymph nodes, lymphatic flow, biliary ducts, and other tissues during surgical procedures. The imaging system 10 provides an adjunctive method for evaluation of tissue perfusion and related tissue-transfer circulation during surgery. The imaging system 10 may utilize NIR excitation laser light having a wavelength of from about 780 nm to about 805 nm and observation range from about 825 nm to about 850 nm. Fluorescence may be provided by a fluorescent dye having matching excitation and emission ranges. The fluorescence light may be detected by an infrared (IR) channel of a camera assembly to produce an IR image. Other channels of the camera assembly may be used to capture white light images of the same scene. Two images, the white light image and the IR image, may be blended and/or combined to produce a composite image.

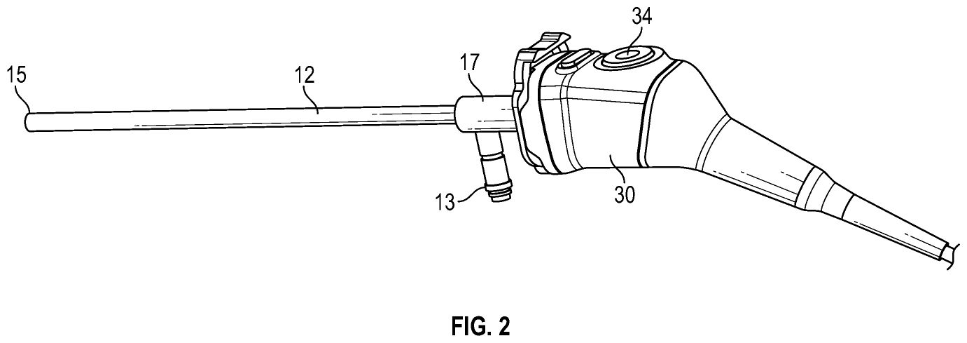

With reference to FIGS. 1 and 2, the imaging system 10 includes an endoscope 12 having a longitudinal shaft 14 with a plurality of optical components (not shown), such as lenses, mirrors, prisms, and the like disposed in the longitudinal shaft 14. The endoscope 12 is coupled to a combined light source 16 via an optical cable 18. The light source 16 may include a white light source (not shown) and an NIR light source (not shown), which may be light emitting diodes or any other suitable light sources. The NIR light source may be a laser or any other suitable light source. The optical cable 18 may include one or more optical fibers for transmitting the white and NIR light, which illuminates the tissue under observation by the endoscope 12. The endoscope 12 collects the reflected white and NIR light and transmits the same to a camera assembly 30, which is coupled to a proximal end portion of the endoscope 12. The endoscope 12 may be any conventional endoscope configured to transmit and collect white and IR light.

The camera assembly 30 is coupled to a camera control unit 20 via a transmission cable 24. The camera control unit 20 is configured to receive the image data signals, process the raw image data from the camera assembly 30, and generate blended white light and NIR images for recording and/or real-time display. The camera control unit 20 also processes the image data signals and outputs the same to a display 26, through any suitable a video output port, such as a DISPLAYPORT™, HDMI®, etc., that is capable of transmitting processed images at any desired resolution, display rates, and/or bandwidth.

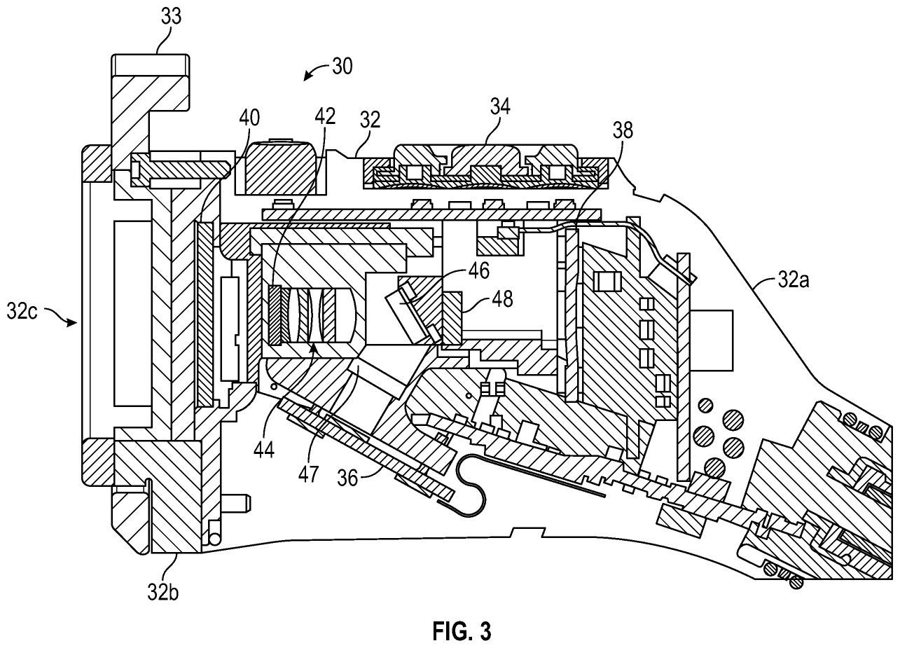

With reference to FIGS. 2 and 3, the endoscope 12 includes a light cable port 13 configured to connect to the optical cable 18. The endoscope 12 includes a distal end portion 15 configured to emit and receive light transmitted therethrough from the light source 16 and to receive light reflected from the environment (e.g., tissue). The reflected light is transmitted through the endoscope and through a proximal end 17 that is coupled to the camera assembly 30.

The camera assembly 30 includes a housing 32 having a proximal end portion 32a and a distal end portion 32b with an opening 32c. The camera assembly 30 includes a connector 33 configured to releasably couple the endoscope 12 to the camera assembly 30, namely, the proximal end 17 to the housing 32. The connector 33 may be a spring-loaded locking connector that locks the endoscope 12 to the camera assembly 30 until released. The camera assembly 30 also includes a user interface 34 having one or more buttons for controlling the camera assembly 30, such as zoom, brightness, etc.

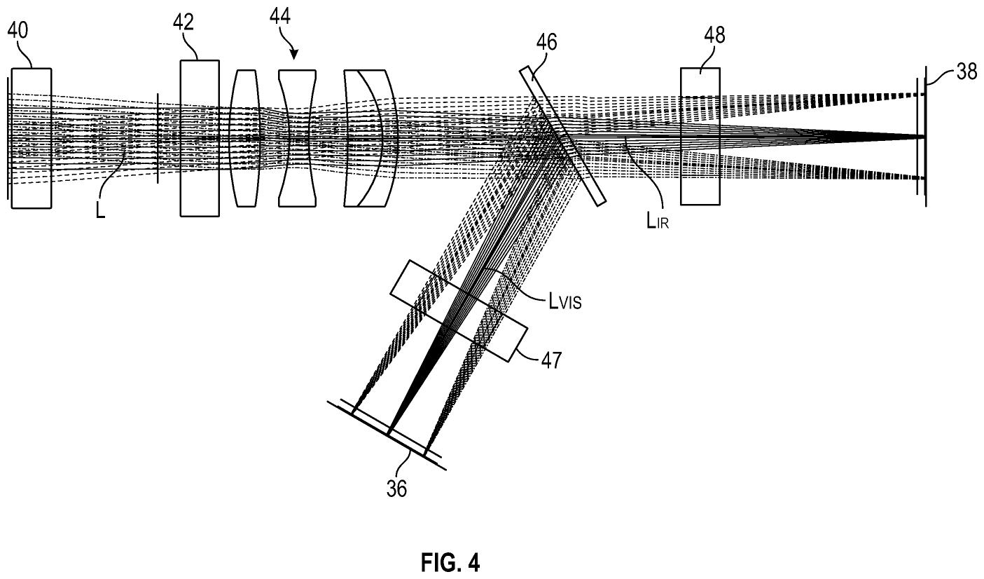

With reference to FIGS. 3 and 4, the camera assembly 30 includes a novel optical design and is configured to separate fluorescence wavelengths from undesired components of the light spectrum to specific sensors. In particular, the camera assembly 30 includes a white (e.g., visible) light (VIS) sensor 36 and an IR sensor 38 and is configured to separate and transmit white light to the VIS sensor 36 and fluorescence IR light to the IR sensor 38. The VIS sensor 36 and the IR sensor 38 may be a complementary metal oxide semiconductor (CMOS) image sensors having any desired resolution, which in embodiments may be 4K, UHD, etc.

The camera assembly 30 includes a window 40 disposed adjacent to the connector 33. The light from the endoscope 12 is transmitted along a light path L through the window 40 to a notch filter 42 that is configured to selectively reject a portion of the received light. In particular, the notch filter 42 is configured to reject the laser wavelength of the light source 16, e.g., from an excitation laser. In embodiments, the notch filter 42 may be configured to reject excitation laser light having a wavelength from about 780 nm to about 805 nm. The notch filter 42 is also configured to passthrough the fluorescent light, which is at a higher wavelength than the excitation laser light, i.e., from about 825 nm to about 850 nm, and visible light which is lower than the excitation laser light, i.e., from about 380 nm to about 700 nm.

The camera assembly 30 also includes a focus group 44 having one or more lenses. The focus group 44 is configured to focus the light on the VIS sensor 36 and the IR sensor 38. This is accomplished by moving the focus group 44 longitudinally along the light path L using any suitable drive mechanism (e.g., piezoelectric actuators). The notch filter 42 may be coupled to the focus group 44 such that the notch filter 42 is also movable along with the focus group 44.

The camera assembly 30 further includes a cold mirror 46, which is a specialized dielectric mirror, which acts as a dichroic filter or beamsplitter, that reflects most or all of the visible light along a VIS light path L_{VIS} while efficiently transmitting IR fluorescence light along IR light path L_{IR}. In particular, the cold mirror 46 is configured to reflect visible light having a wavelength from about 380 nm to about 700 nm and to transmit IR fluorescence light having a wavelength from about 825 nm to about 850 nm. The cold mirror 46 may be disposed at any incidence angle, which may be from about 10° to about 80° relative to the light path L. The cold mirror 46 may include a plurality of dielectric coatings disposed in a multi-layer configuration.

The IR light passing through the cold mirror 46 is then passed through a bandpass filter 48 to further eliminate any of the visible light spectrum that may have passed through the cold mirror 46. The bandpass filter 48 is an optical filter that is configured to selectively transmit a portion of the spectrum, in this case, fluorescence IR light having a wavelength from about 825 nm to about 850 nm, while rejecting all other wavelengths, i.e., visible light and IR laser light. The IR light passes through the bandpass filter 48 and is received by the IR sensor 38, which then outputs image data corresponding to received IR light.

Regarding the visible light that is reflected by the cold mirror 46, the visible light is transmitted along the VIS light path L_{VIS} at the desired incidence angle that is transverse to the light path L and the IR light path L_{IR}. The visible light passes through a hot mirror 47, which is a specialized dielectric mirror, which also acts as a dichroic filter or beamsplitter, that reflects most or all of the IR fluorescence light having a wavelength from about 825 nm to about 850 nm. The hot mirror 47 also efficiently transmits visible light having a wavelength from about 380 nm to about 700 nm along the VIS light path L_{VIS} toward the VIS sensor 36. The hot mirror 47 is disposed substantially perpendicularly to the VIS light path L_{VIS}, such that IR light is reflected in a reverse direction along the VIS light path L_{VIS} toward the cold mirror 46. As used herein, the term “substantially perpendicular” denotes a relative configuration and is +/−5° from true perpendicular of 90°.

The above disclosed optical configuration of the camera assembly 30 separates and filters visible and IR light along divergent paths, allowing for use of two separate sensors (i.e., the VIS sensor 36 and the IR sensor 38). This allows for operating the VIS sensor 36 and the IR sensor 38 at highest possible frame rates for each of the visible and IR light channels, resulting in a smoother video stream while combining visible and IR images. Furthermore, filtering of the light through the notch filter 42, the cold mirror 46, and the bandpass filter 48 allows for using a highly sensitive IR sensor 38, which normally would not be possible due to higher intensity IR light present in the reflect light. Using higher sensitivity IR sensors 38 is beneficial in identifying critical tissue elements, such as cancer cells. Another advantage of the camera assembly 30 of the present disclosure is the ability to use the camera assembly 30 with any conventional endoscope since all the light filters and mirrors are disposed at the camera assembly 30.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

1. A camera assembly comprising:
a housing having an opening configured to couple to an endoscope and to receive a combined light entering the housing along a combined light path, the combined light including visible light and infrared fluorescence light;
a cold mirror configured to split the combined light into the visible light along a visible light path and the infrared fluorescence light along an infrared light path;
a visible light sensor configured to receive the visible light and to generate visible light image data;
an infrared sensor configured to receive the infrared fluorescence light and to generate infrared fluorescence image data; and
a hot mirror disposed along the visible light path between the cold mirror and the visible light sensor and at an incidence angle that is substantially perpendicular to the visible light path, the hot mirror configured to transmit the visible light and to reflect the infrared fluorescence light.

2. The camera assembly according to claim 1, further comprising:
a notch filter disposed adjacent the opening, such that the combined light entering the opening passes through the notch filter, the notch filter configured to remove excitation laser light from the combined light.

3. The camera assembly according to claim 2, further comprising:
a focus group including a plurality of lenses, the focus group disposed between the opening and the cold mirror and movable along the combined light path.

4. The camera assembly according to claim 3, wherein the notch filter is coupled to the focus group and is movable along with the focus group.

5. (canceled)

6. (canceled)

7. The camera assembly according to claim 1, further comprising:
a bandpass filter disposed along the infrared light path and between the cold mirror and the infrared sensor, the bandpass filter configured to transmit only the infrared fluorescence light to the infrared sensor.

8. An endoscope assembly comprising:
an endoscope including a proximal end portion, a distal end portion, and a light port configured to couple to a light source, the endoscope configured to receive a combined light including visible light and infrared fluorescence light; and
a camera assembly comprising:
a housing having an opening configured to couple to the distal end portion of the endoscope and to receive the combined light entering the housing along a combined light path;
a cold mirror configured to split the combined light into the visible light along a visible light path and the infrared fluorescence light along an infrared light path;
a visible light sensor configured to receive the visible light and to generate visible light image data;
an infrared sensor configured to receive the infrared fluorescence light and to generate infrared fluorescence image data; and
a hot mirror disposed along the visible light path between the cold mirror and the visible light sensor and at an incidence angle that is substantially perpendicular to the visible light path, the hot mirror configured to transmit the visible light and to reflect the infrared fluorescence light.

9. The endoscope assembly according to claim 8, wherein the camera assembly further includes:
a notch filter disposed adjacent the opening, such that the combined light entering the opening passes through the notch filter, the notch filter configured to remove excitation laser light from the combined light.

10. The endoscope assembly according to claim 9, wherein the camera assembly further includes:
a focus group including a plurality of lenses, the focus group disposed between the opening and the cold mirror and movable along the combined light path.

11. The endoscope assembly according to claim 10, wherein the notch filter is coupled to the focus group and is movable along with the focus group.

12. (canceled)

13. (canceled)

14. The endoscope assembly according to claim 8, wherein the camera assembly further includes:
a bandpass filter disposed along the infrared light path and between the cold mirror and the infrared sensor, the bandpass filter configured to transmit only the infrared fluorescence light to the infrared sensor.

15. An imaging system comprising:
a combined light source configured to output visible light and excitation laser light;
an endoscope including a proximal end portion, a distal end portion, and a light port configured to couple to the light source, the endoscope configured to receive a combined light including visible light and infrared fluorescence light;
a camera assembly comprising:
a housing having an opening configured to couple to the distal end portion of the endoscope and to receive the combined light entering the housing along a combined light path;
a cold mirror configured to split the combined light into the visible light along a visible light path and the infrared fluorescence light along an infrared light path;
a visible light sensor configured to receive the visible light and to generate visible light image data;
an infrared sensor configured to receive the infrared fluorescence light and to generate infrared fluorescence image data; and
a hot mirror disposed along the visible light path between the cold mirror and the visible light sensor and at an incidence angle that is substantially perpendicular to the visible light path, the hot mirror configured to transmit the visible light and to reflect the infrared fluorescence light; and
a camera control unit coupled to the camera assembly, the camera control unit configured to output a combined image including the visible light image data and the infrared fluorescence image data.

16. The imaging system according to claim 15, wherein the camera assembly further includes:
a notch filter disposed adjacent the opening, such that the combined light entering the opening passes through the notch filter, the notch filter configured to remove the excitation laser light from the combined light.

17. The imaging system according to claim 16, wherein the camera assembly further includes:
a focus group including a plurality of lenses, the focus group disposed between the opening and the cold mirror and movable along the combined light path.

18. The imaging system according to claim 17, wherein the notch filter is coupled to the focus group and is movable along with the focus group.

19. (canceled)

20. The imaging system according to claim 15, wherein the camera assembly further includes:
a bandpass filter disposed along the infrared light path and between the cold mirror and the infrared sensor, the bandpass filter configured to transmit only the infrared fluorescence light to the infrared sensor.
